(19) United States
(12) Patent Application Publication
(10) Pub. No.: US 2022/0202599 A1
(43) Pub. Date: Jun. 30, 2022

(54) GAP-ADJUSTABLE/ELIMINABLE SHOCK ABSORPTION STRUCTURE

(71) Applicant: Ken Dall Enterprise Co., Ltd., New Taipei City (TW)

(72) Inventors: Chia-Pao CHENG, New Taipei City (TW); Hsiang-Ming WU, New Taipei City (TW); Chih-Hsuan LIANG, New Taipei City (TW)

(21) Appl. No.: 17/243,592

(22) Filed: Apr. 29, 2021

(30) Foreign Application Priority Data
Dec. 28, 2020 (TW) 109217180

(51) Int. Cl. A61F 2/66

(52) U.S. Cl. CPC A61F 2/6607; A61F 2002/5003

(57) ABSTRACT

A gap-adjustable/eliminable shock absorption structure includes a shock-absorbing ankle body having a thread adjustment portion. An elastic body and a driving piston are provided on a spring carrier pivotally mounted inside the shock-absorbing ankle body. The driving piston is connected, through screwing, to an inverted T-shaped connector. A cylinder extending from the driving piston is fit into a friction bushing, such that a surface of the cylinder is closely fit to an inside surface of the friction bushing. The friction bushing has an oblique outside surface that is closely fit to an internal wall of the shock-absorbing ankle body. The thread adjustment portion receives a fastening ring and an adjusting ring to screw thereto such that fine adjustment is achieved through displacement caused by rotation of the adjusting ring to set up tight engagement with the friction bushing as being closely fit to the surface of cylinder surface.

### TECHNICAL FIELD OF THE INVENTION

The present invention relates to a shock absorption structure of use with an artificial limb, and more particularly to an arrangement in which a friction bushing includes an arrangement of oblique surface that matches an inclined surface of a corresponding structure, in combination with a fastening ring and an adjusting ring, to achieve fine adjustment of a position of the friction bushing, so as to make no gap present between the friction bushing and the driving piston the cylinder surface and maintaining a state of being completely and closely fit therebetween thereby extending the service life of the entirety of the shock absorption structure of the artificial limb

### DESCRIPTION OF THE PRIOR ART

Artificial joints that are available currently are diversified in respect of the structural arrangements thereof, and this leads to complicated operation of a cushioning structure. For example, Chinese Patent CN211271428 U provides a vacuum suction twisting shock-absorbing connector, of which an internal structure is such that when a pivoting member is shifted upwards, an air inlet opening conducts air to flow to an upper air chamber to form vacuum, and a lower air chamber is operable, in combination with an air discharge opening, to discharge; and when the pivoting member is shifted downward, air flows from the upper air chamber to the lower air chamber to form vacuum, so as to allow the pivoting member to stably operate in a carrier seat. Allowing the upper and lower air chambers to alternately suck in air make it possible for a sleeve to fast form vacuum in the carrier seat in which the compartment and the discharge opening are formed. Although an elastic element is included for the purpose of cushioning, use of an extra vacuum suction function for operation is included.

However, most of the artificial limb devices often involves an elastic element for use in combination with cushioning. When the elastic element is used for the purposes of cushioning, the structure is simple and easy in respect of replacement and servicing. However, since the parts are mostly made of metallic materials, although smooth operation may be ensured by adding lubricants, yet there may still be gaps generated by wear and abrasion among the metallic parts. This may cause shaking or instability in the operation of the artificial limb as a whole. Such a situation cannot be resolved unless the entire structure is replaced.

### SUMMARY OF THE INVENTION

The primary objective of the present invention is to provide a structure, in which a fastening ring and an adjusting ring are provided for operation of fine adjustment to make a friction bushing completely and closely fit to a driving piston to maintain stability of cushioning so as to extend a service life of a shock absorption structure, which is applicable to resolve a technical problem for innovation and breakthrough in developing a shock absorption structure for an artificial limb and having easy operation for adjustment and exhibiting a bettered practical utilization and also helping extend the service life thereof.

The technical feature of the present invention is mainly that a gap-adjustable/eliminable shock absorption structure, which comprises a shock-absorbing ankle body, which includes a thread adjustment portion protruding from a top end thereof and a primary pivoting hole formed in a side edge of a bottom end thereof; a spring carrier, which includes a secondary pivoting hole in alignment with the primary pivoting hole to receive a pivot axle to extend therethrough to have the spring carrier pivotally mounted in an interior of the shock-absorbing ankle body, an elastic body and a driving piston being arranged, in sequence, on the spring carrier, the driving piston having a top end connected by means of a thread formed in an interior thereof to an inverted T-shaped connector through screwing engagement therebetween to form a connected condition, wherein a friction bushing is fit over a cylinder extending from the driving piston, and the cylinder has a cylinder surface that is closely fit to an inside surface of the friction bushing, the friction bushing having an outside surface that is made an inclined surface matching with and is closely fit to an internal wall of the shock-absorbing ankle body, the thread adjustment portion receiving, in sequence from bottom to top, a fastening ring and an adjusting ring to screw thereto for connecting, wherein the adjusting ring is rotatable for displacement upwards or downwards to achieve fine adjustment, so as to set in tight contact engagement with the friction bushing to maintain a completely and closely fit state with respect to the cylinder surface to extend a service life of the driving piston.

A secondary technical purpose is that the outside surface of the friction bushing is of a convergent arrangement in a manner of tapering from an upper end to a lower end.

Another secondary technical purpose is that the driving piston is of an arrangement of polygonal shape to prevent inappropriate rotation of the driving piston that causes deviation of sole positioning and causes support instability during walking.

Based on the above unique design of the present invention, as compared with the prior art techniques, the present invention uses the inclined or oblique surface arrangement of the friction bushing set in contact with an inclined surface of a corresponding structure, to achieve fine adjustment through upward or downward displacement by means of adjustment made on the fastening ring and the adjusting ring, so as to eliminate the situation of gap generated in the shock absorption structure by abrasion and wear, and thus extending the service life thereof.

### BRIEF DESCRIPTION OF THE DRAWINGS

FIG. 1 is a perspective view of the present invention.

FIG. 2 is an exploded view of the present invention shown in FIG. 1.

FIG. 3 is a schematic view showing an initial, uncompressed state of the present invention.

FIG. 4 is a cross-sectional view showing the initial, uncompressed state of the present invention.

FIG. 5 is a schematic view showing a compressed state of the present invention.

FIG. 6 is cross-sectional view showing the compressed state of the present invention.

FIG. 7 is a perspective view of a first alternative embodiment of the present invention.

FIG. 8 is an exploded view of the first alternative embodiment of the present invention shown in FIG. 7.

FIG. 9 is a schematic view, partially sectioned, of the first alternative embodiment of the present invention.

FIG. 10 is a perspective view of a second alternative embodiment of the present invention.

FIG. 11 is an exploded view of the second alternative embodiment of the present invention shown in FIG. 10.

FIG. 12 is a schematic view, partially sectioned, of the second alternative embodiment of the present invention.

### DETAILED DESCRIPTION OF THE PREFERRED EMBODIMENT

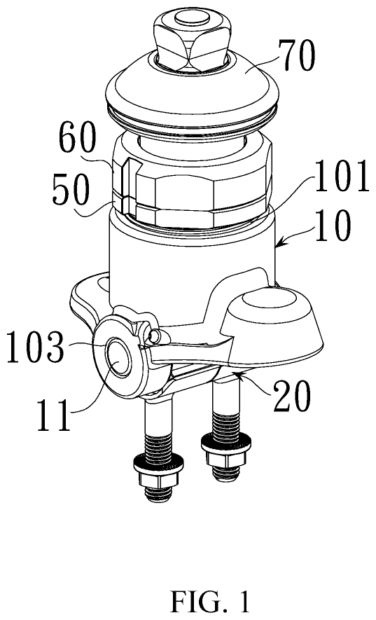

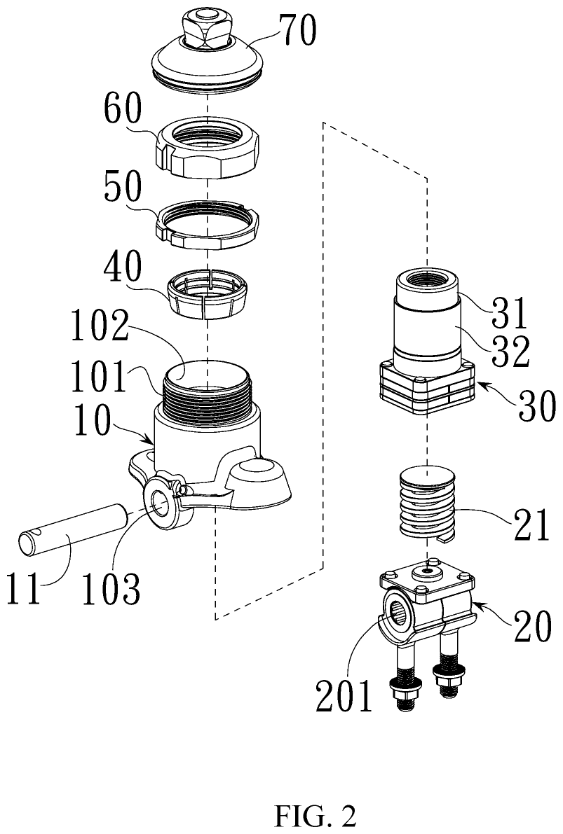

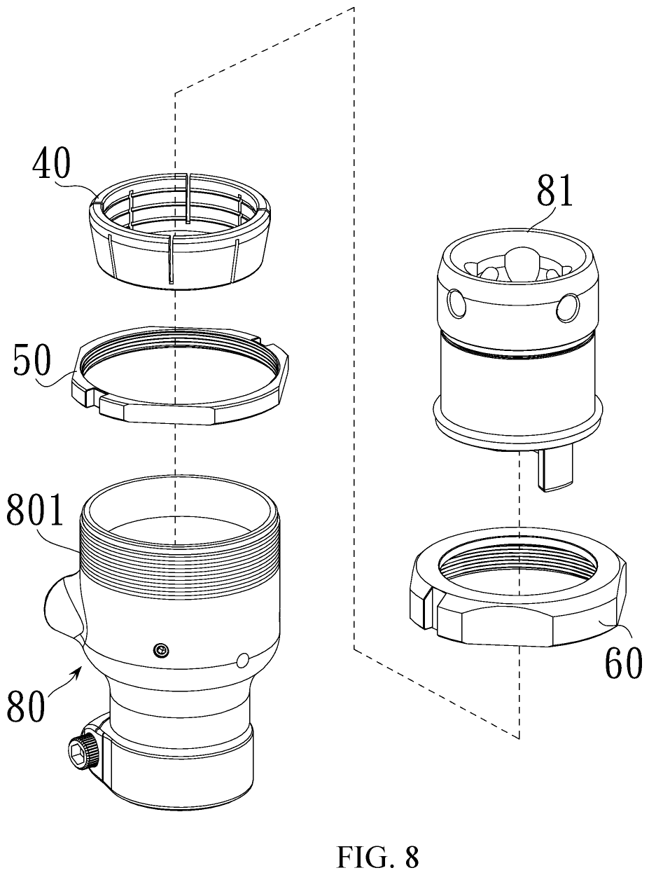

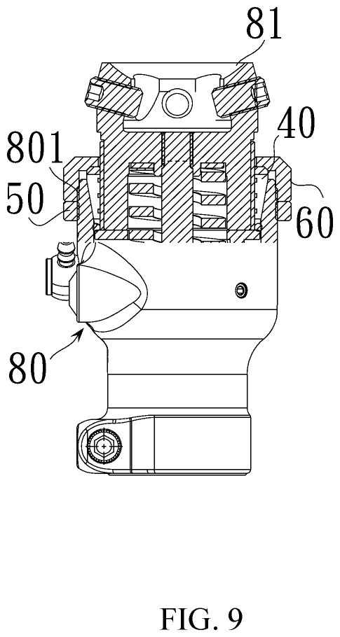

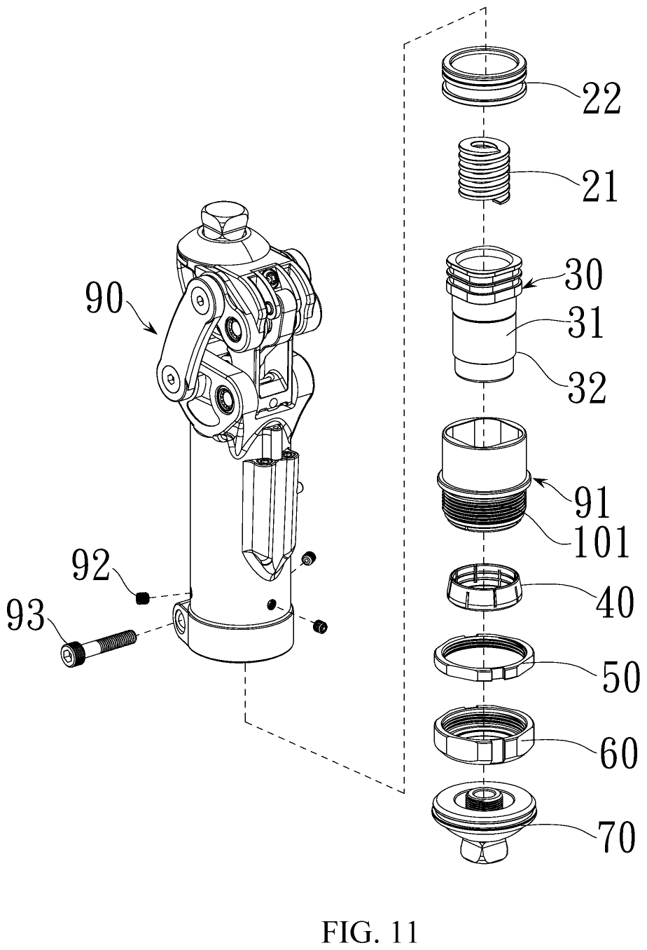

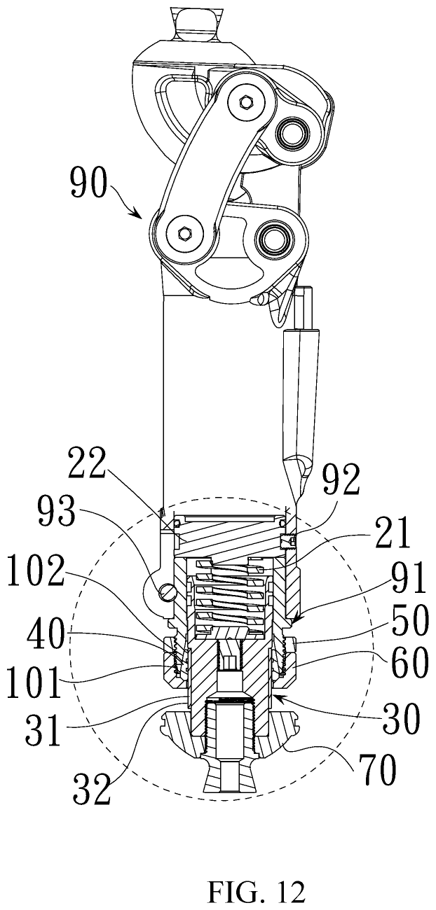

A preferred feasible embodiment according, generally, to the present invention will be described, in detail, with reference to FIGS. 1-12, for better understanding of the present invention. The present invention provides a gap-adjustable/eliminable shock absorption structure, which comprises a shock-absorbing ankle body (10), which is formed with a thread adjustment portion (101) protruding from a top end thereof and a primary pivoting hole (103) formed in a side edge of a bottom end of the shock-absorbing ankle body (10); a spring carrier (20), which includes a secondary pivoting hole (201) generally aligned with the primary pivoting hole (103) to receive a pivot axle (11) to extend therethrough in order to have the spring carrier (20) pivotally mounted in an interior of the shock-absorbing ankle body (10) and an elastic body (21) and a driving piston (30) that are arranged, in sequence, on the spring carrier (20) to cushion a downward pressing force. Here, the downward pressing force is referred to a force resulting from the weight of a user and successive movements of walking that the artificial limb has to bear. The driving piston (30) has a top end that is connected, through an internal thread formed in an inside surface thereof, with an inverted T-shaped connector (70) that is formed with an external thread to form a connected condition.

A friction bushing (40) is fit over a cylinder (31) extending from the driving piston (30). The cylinder (31) has a cylinder surface (32) that is closely fit to an inside surface of the friction bushing (40), while an outside surface of the friction bushing (40) is of a convergent arrangement in a manner of tapering from an upper end to a lower end.

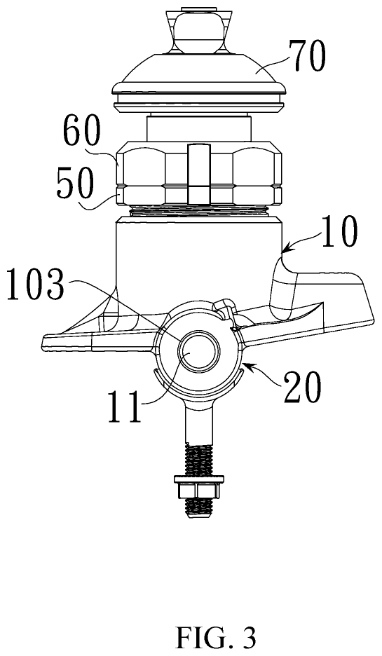

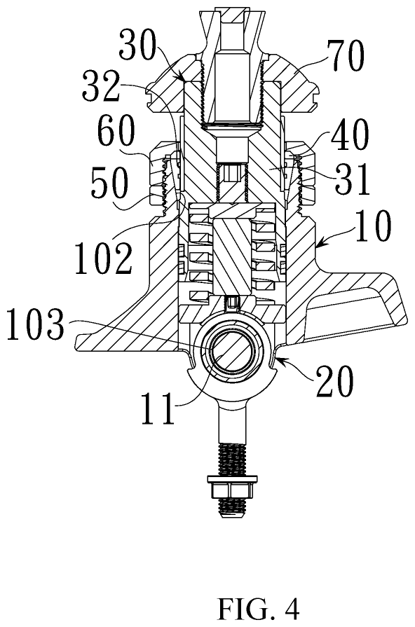

Referring to FIGS. 3 and 4, which show an initial, uncompressed state, it can be seen that the outside surface of the friction bushing (40) is an oblique or inclined surface, which matches an arrangement of an internal wall (102) of the shock-absorbing ankle body (10) that is formed as an inclined surface to provide close fitting therebetween. The thread adjustment portion (101) receives, in sequence from bottom to top, a fastening ring (50) and an adjusting ring (60) connected thereto as being screwed therewith.

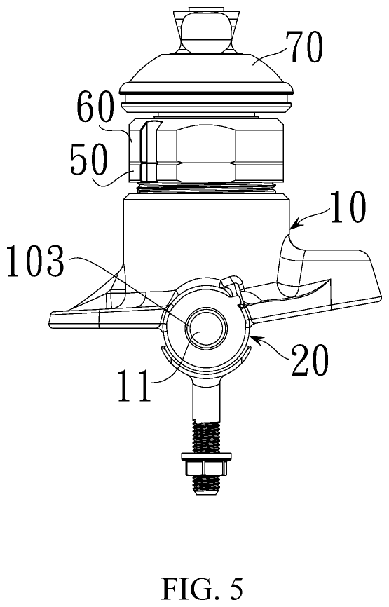

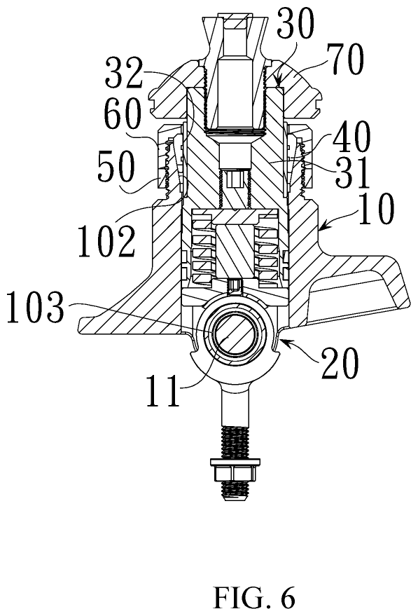

Further referring to FIGS. 5 and 6, when the inverted T-shaped connector (70) is caused to drive the driving piston (30) to move downwards, a compressed state is formed. When the compressed state and the uncompressed state are switched repeatedly in an extended period of time, since metallic materials that make the structure itself would suffer abrasion and wear to generate a gap therebetween, the gap would causes oscillation or shaking, or un-smoothness and noise, during state switching.

Thus, the adjusting ring (60) is made rotatable for displacement upwards or downwards to achieve fine adjustment, so as to ensure and maintain the friction bushing (40) in a state of being completely and closely fit to the cylinder surface (32), this helping extend the service life of the driving piston (30). Further, the driving piston (30) is of a design of polygonal shape to help prevent deviation of sole positioning, resulting from inappropriate, undesired rotation of the driving piston (30), which causes support instability during walking.

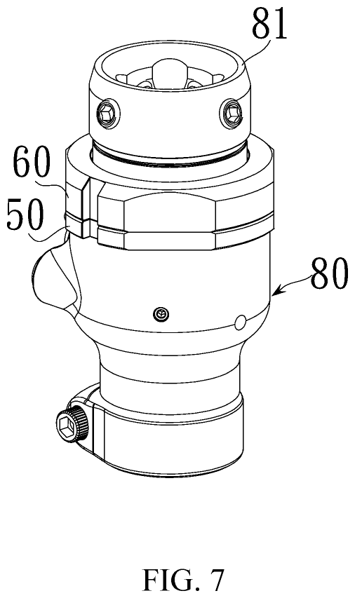

Further referring to FIGS. 7-9, another embodiment of the present invention is shown. A shock-absorbing body (80) is formed with an interior space for receiving a driving portion (81) to dispose therein for connecting. A friction bushing (40) is arranged between the driving portion (81) and the shock-absorbing body (80). The shock-absorbing body (80) is formed a thread portion (801) protruding from a top end thereof. The thread portion (801) receives, in sequence from bottom to top, a fastening ring (50) and an adjusting ring (60) connected thereto as being screwed therewith. The adjusting ring (60) is made rotatable for displacement upwards or downwards to achieve fine adjustment, so as to ensure and maintain the friction bushing (40) in a state of being completely and closely fit between the driving portion (81) and the shock-absorbing body (80), so as to help extend the service life of the driving portion (81).

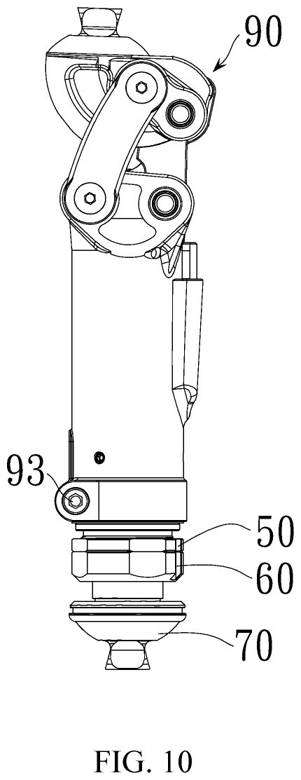

Further referring to FIGS. 10-12, a further embodiment of the present invention is shown, which is provided generally as a structure arranged below an artificial joint (90) to be used for purposes of connecting. A driving piston (30) is provided, on a top end thereof, with an elastic base (22) that is for constraining a structure that is disposed in an interior of the driving piston (30) for cushioning purposes and is positioned by a plurality of fastening bolts (92) penetrating through the artificial joint (90) to engage the elastic base (22). Further provided is a socket head bolt (93) extending through the artificial joint (90) and engaging an outside surface of the piston barrel (91) to form constraint thereto.

The piston barrel (91) is structured to allow a top portion thereof to receive the driving piston (30) to insert therein from a top thereof and connect thereto. The piston barrel (91) is formed, on an outside surface of a lower portion thereof, with a thread adjustment portion (101). A friction bushing (40) is fit over a cylinder (31) extending from the driving piston (30). The cylinder (31) has a cylinder surface (32) that is closely fit to an inside surface of the friction bushing (40), while an outside surface of the friction bushing (40) is made an inclined surface that matches with and is closely fit to an internal wall (102) of the piston barrel (91). The thread adjustment portion (101) receives, in sequence from bottom to top, a fastening ring (50), an adjusting ring (60), and an inverted T-shaped connector (70) connected thereto as being screwed therewith. The adjusting ring (60) is rotatable for displacement upwards or downwards to achieve fine adjustment, so as to tightly contact the friction bushing (40) to ensure and maintain a completely and closely fit state with respect to the cylinder surface (32), helping extend the service life of the driving piston (30).

In summary, the present invention provides a gap-adjustable/eliminable shock absorption structure, which includes an arrangement of a friction bushing (40) in an interior of a shock-absorbing ankle body (10) to reduce and cushion an external force applied thereto and also allowing for fine adjustment made through the fastening ring (50) and the adjusting ring (60) to set the friction bushing (40) in a state of being completely and closely fit to the driving piston (30) in order to extend the service life of the entire structure.

I claim:

1. A gap-adjustable/eliminable shock absorption structure, comprising: a shock-absorbing ankle body, which includes a thread adjustment portion protruding from a top end thereof and a primary pivoting hole formed in a side edge of a bottom end thereof; a spring carrier, which includes a secondary pivoting hole in alignment with the primary pivoting hole to receive a pivot axle to extend therethrough to have the spring carrier pivotally mounted in an interior of the shock-absorbing ankle body, an elastic body and a driving piston being arranged, in sequence, on the spring carrier, the driving piston having a top end connected by means of a thread formed in an interior thereof to an inverted T-shaped connector through screwing engagement therebetween to form a connected condition, wherein a friction bushing is fit over a cylinder extending from the driving piston, and the cylinder has a cylinder surface that is closely fit to an inside surface of the friction bushing, the friction bushing having an outside surface that is made an inclined surface matching with and is closely fit to an internal wall of the shock-absorbing ankle body, the thread adjustment portion receiving, in sequence from bottom to top, a fastening ring and an adjusting ring to screw thereto for connecting, wherein the adjusting ring is rotatable for displacement upwards or downwards to achieve fine adjustment, so as to set in tight contact engagement with the friction bushing to maintain a completely and closely fit state with respect to the cylinder surface to extend a service life of the driving piston.

2. The gap-adjustable/eliminable shock absorption structure according to claim 1, wherein the outside surface of the friction bushing is of a convergent arrangement in a manner of tapering from an upper end to a lower end.

3. The gap-adjustable/eliminable shock absorption structure according to claim 1, wherein the driving piston is of an arrangement of polygonal shape to prevent inappropriate rotation of the driving piston that causes deviation of sole positioning and causes support instability during walking.

4. A gap-adjustable/eliminable shock absorption structure, comprising: a shock-absorbing body that is formed with an interior space for receiving a driving portion to dispose therein for connecting, a friction bushing being arranged between the driving portion and the shock-absorbing body, the shock-absorbing body being formed a thread portion protruding from a top end thereof, the thread portion receiving, in sequence from bottom to top, a fastening ring and an adjusting ring, connected thereto as being screwed therewith, wherein the adjusting ring is rotatable for displacement upwards or downwards to achieve fine adjustment, so as to ensure and maintain the friction bushing in a state of being completely and closely fit between the driving portion and the shock-absorbing body, so as to extend a service life of the driving portion.

5. A gap-adjustable/eliminable shock absorption structure, which is arranged below an artificial joint to be used for connecting, comprising: a driving piston, which is provided, on a top end thereof, with an elastic base that is for constraining a structure that is disposed in an interior of the driving piston for cushioning purposes and is positioned by a plurality of fastening bolts penetrating through the artificial joint to engage the elastic base; a piston barrel that receives a driving piston to insert therein from a top thereof and connect thereto, the piston barrel being formed, on an outside surface of a lower portion thereof, with a thread adjustment portion, wherein a friction bushing is fit over a cylinder extending from the driving piston, the cylinder having a cylinder surface that is closely fit to an inside surface of the friction bushing, the friction bushing having an outside surface that is made an inclined surface matching and closely fit to an internal wall of the piston barrel, the thread adjustment portion receiving, from top to bottom in a manner of being screwed therewith, a fastening ring, an adjusting ring, and an inverted T-shaped connector, wherein the adjusting ring is rotatable for displacement upwards or downwards to achieve fine adjustment, so as to tightly contact the friction bushing to ensure and maintain a completely and closely fit state with respect to the cylinder surface to extend a service life of the driving piston.
